(19) United States
(12) Patent Application Publication
(10) Pub. No.: US 2022/0395435 A1
(43) Pub. Date: Dec. 15, 2022

(54) Fibrous Product For Medicinal, Cosmetic or Dermatologic Use

(71) Applicant: Schweitzer-Mauduit International, Inc., Alpharetta, SC (US)

(72) Inventors: Cedric Rousseau, Le Mans (FR); Doriane Bigot, Le Mans (FR)

(21) Appl. No.: 17/773,402

(22) PCT Filed: Oct. 30, 2020
(86) PCT No.: PCT/US2020/058294

§ 371 Date: Apr. 29, 2022

Related U.S. Application Data
(60) Provisional application No. 62/929,377, filed on Nov. 1, 2019.

(51) Int. Cl. A61K 8/02; A61K 9/00; A61K 9/70; A61K 8/9789; A61K 36/185; A61Q 19/00

(52) U.S. Cl. CPC A61K 8/0212; A61K 9/0087; A61K 9/7007; A61K 8/9789; A61K 36/185; A61Q 19/00; A61K 2236/50

(57) ABSTRACT

A medicinal, cosmetic or dermatologic product is described made from a reconstituted cannabis material. The reconstituted cannabis material forms a sheet-like layer that can be shape configured to cover a portion of a user's body. The product can be used as a face mask, as a wound dressing, or the like.

### RELATED APPLICATIONS

The present application is based upon and claims priority to Provisional Patent Application Ser. No. 62/929,377, having a filing date of Nov. 1, 2019, and which is incorporated herein by reference.

### BACKGROUND

Today, materials originating from a plant are used in many applications. If the use of plant based products is intended, e.g., to color items or hair or food or skin because of a preference for natural ingredients, it usually requires specific conditions, such as solvent types, moisture content, temperatures, pH, etc., and can take a considerable time until the final result. Indeed, when a plant product is applied, e.g., in form of a powder, e.g., on hair or skin, two things take place: an extraction of the substance(s) and its fixation on the hair or skin. Actually, it requires a long contact time to obtain a significant result. Moreover, hair or skin can be damaged by substances in the plant, such as traces of heavy metals, pesticides, or polyphenols. In addition, the quantity of the substance(s) of interest is not necessarily consistent from one application to another due to the variability of the natural ingredient(s). Hence, applying the same amount of a hair coloring product may lead to different results.

The foregoing example is equally true for other uses of plant materials, such as cosmetic, medicinal or dermatologic uses. So far, many substances from plant materials cannot be used in medicinal or dermatologic applications due to the time required for the substance to unfold its effect. In addition, in many applications, the concentration of a substance necessary for a certain medicinal or dermatologic effect cannot be achieved or else the administration would become difficult. Plant materials may release an insufficient amount of substance(s) and/or have a low release rate. Sublingual administration is very often impossible for the same reason. Moreover, in medicinal or dermatologic uses it may be the case that only the combination of more substances or a complex extract of the plant(s) leads to the desired effect. Sometimes it is not entirely known how this combination works and what substances are necessary to achieve the desired effect. In such cases it is desirable to use most or substantially all substances contained in the respective plant(s). On the other hand, it may be desirable to separate certain desired substances from certain undesired substances such as potentially toxic components of the plant.

There is still a need to improve products originating from plant materials for medicinal or cosmetic or dermatologic use. In particular, it is desirable to control the amount of substances originating from plant materials as well as conditions and time needed to achieve a desired medicinal or cosmetic or dermatologic effect.

### SUMMARY

The invention relates to a product comprising plant materials as raw materials. In particular, the product may comprise a fibrous plant product and optionally a plant extract. The fibrous plant product may comprise solid parts of a plant and the plant extract may comprise substances extracted from a plant. The fibrous plant product may form a layer on which the plant extract is applied. The plant extract can form a second layer or at least partially enter or penetrate into the fibrous plant product. According to the invention it is possible to first separate substances from one or more plants and combine one or more of the remaining or separated substances subsequently.

The plant can be separated into a plant extract and a fibrous plant product. Subsequently the fibrous plant product and the plant extract are combined to obtain a reconstructed or reconstituted version of the original plant with improved properties. For example, certain substances of the original plant may be easily water-soluble and others not. In this way, one can accelerate or even control the release or extraction rate of certain substances to achieve a medicinal or cosmetic or dermatologic effect. Also, there can be higher concentrations of certain or all substances as compared to the natural plant.

In accordance with the present disclosure, the medicinal, cosmetic or dermatologic product is formed at least in part from cannabis components. For instance, in one embodiment, the product comprises a sheet-like layer comprising a reconstituted cannabis material. The reconstituted cannabis material can contain extracted cannabis fibers obtained from cannabis leaves, cannabis hurds or stems, cannabis buds, cannabis flowers, cannabis seeds or shells, or mixtures thereof. Optionally, the extracted cannabis fibers can be combined with web building fibers to produce the reconstituted cannabis material. The web building fibers, for instance, may comprise delignified cellulosic fibers, such as wood pulp fibers, hemp fibers, or the like. In one aspect, the product has a shape configured to cover a portion of a user's body.

Optionally, the sheet-like layer can be treated with a plant extract. In one embodiment, the plant extract can be obtained entirely from the cannabis materials or components used to form the reconstituted cannabis material. In other embodiments, the plant extract can contain a cannabis extract in combination with other plant extracts. In still another embodiment, the plant extract can be obtained solely from other plant extracts without containing a cannabis extract.

The plant extract can be impregnated into the reconstituted cannabis material. Alternatively, the plant extract can form a layer located on the sheet-like layer made from the reconstituted cannabis material. The plant extract can comprise a fluid, a gel, a slurry, or a powder. Of particular advantage, it was discovered that the reconstituted cannabis material of the present disclosure is particularly well suited to applying plant extracts to a user's skin.

The amount of water soluble components contained in the reconstituted cannabis material can vary depending upon various factors and whether an extract has been applied to the material. In one aspect, the reconstituted cannabis material contains water soluble cannabis components in an amount less than about 10% by weight, such as in an amount less than about 8% by weight, such as in an amount less than about 5% by weight. In another aspect, the reconstituted cannabis material can contain water soluble cannabis components in an amount greater than about 8% by weight, such as in an amount greater than about 10% by weight, such as in an amount greater than about 12% by weight, such as in an amount greater than about 15% by weight, and generally in an amount less than about 60% by weight, such as in an amount less than about 30% by weight.

As described above, in one embodiment, the reconstituted cannabis material can contain web building fibers. The web building fibers can comprise delignified cellulose fibers. In one aspect, the web building fibers comprise wood pulp fibers, such as softwood fibers, hardwood fibers or a mixture of softwood fibers and hardwood fibers. Other web building fibers that may be used include hemp fibers, flax fibers, abaca fibers, bamboo fibers, coconut fibers, ramie fibers, jute fibers, and mixtures thereof. The web building fibers can be present in the reconstituted cannabis material in an amount greater than about 15% by weight, such as in an amount greater than about 18% by weight, such as in an amount greater than about 20% by weight, and generally in an amount less than about 60% by weight, such as in an amount less than about 40% by weight, such as in an amount less than about 30% by weight, such as in an amount less than about 28% by weight, such as in an amount less than about 27% by weight.

In one aspect, the reconstituted cannabis material contains wood pulp fibers in an amount from about 15% to about 25% by weight. In an alternative embodiment, the reconstituted cannabis material can contain hemp fibers or hemp pulp in an amount from about 30% to about 55% by weight, such as in an amount from about 35% to about 45% by weight.

The fibers used to produce the reconstituted cannabis material can optionally be refined prior to forming the sheet-like layer. For example, the cannabis components can optionally be combined with web building fibers and the resulting fiber furnish can be refined in an amount greater than about 40° SR, such as greater than about 60° SR, such as greater than about 65° SR, such as greater than about 70° SR, such as greater than about 75° SR, and generally less than about 90° SR, such as less than about 85° SR, such as less than about 80° SR.

In one aspect, the reconstituted cannabis material includes from about 20% to about 50% by weight extracted cannabis leaves, from about 20% to about 50% by weight extracted cannabis buds and/or flowers, and from about 15% to about 55% by weight web building fibers.

The medicinal, cosmetic or dermatologic product of the present disclosure can also contain a humectant. The humectant can comprise glycerol, propylene glycol, or mixtures thereof. The humectant can be added directly to the reconstituted cannabis material or can be added to a plant extract that is applied to the reconstituted cannabis material. The humectant can be present in the reconstituted cannabis material in an amount of from about 2% to about 25% by weight.

The reconstituted cannabis material can generally have a basis weight of from about 25 gsm to about 100 gsm, such as from about 40 gsm to about 85 gsm.

Many different medicinal, cosmetic or dermatologic products can be formed from the sheet-like layer comprising the reconstituted cannabis material. In one embodiment, for instance, the product is a wound dressing. Alternatively, the product can be a mask that is placed over a user's face. The mask, for instance, can include a pair of opposing eye openings and a mouth opening. The mask can also include an opening or score lines for a nose. The sides of the mask can also touch the user's face. In addition to a user's face, it should be understood that the product of the present disclosure can be applied to any suitable area of the body for general wellness and healing and/or for applying a personal care composition to the skin of a user.

The present disclosure also relates to a method for producing a product. The method may comprise the steps of:
a) extracting one or more substances of at least one plant to obtain a plant extract, the at least one plant comprises cannabis components as described above;
b) separating the plant extract from the at least partially fibrous residue;
c) optionally refining the residue;
d) preparing a sheet like product from at least a part of the residue;
e) optionally concentrating and/or purifying and/or aromatizing the plant extract;
f) optionally applying the plant extract of step b) or e) to the sheet of step d); and
g) optionally drying the product of step f).

It is also possible to select one or more substances or parts from the residue before a product is prepared in step d). Step e) optionally also comprises the selection of certain substances and the filtering of undesired substances. The selection of plants is similar to the respective discussion relating to the product.

In step a) a solvent can be used to extract the one or more substances. A solvent can be any known solvent, such as a polar protic, apolar protic, polar aprotic, apolar aprotic solvent. Also a combination of solvents can be used. The one or more solvents can be determined based on the plant(s) to be processed and the substance(s) to be extracted. Alternatively or in addition to a solvent, extracting the one or more substances can be achieved by mechanical force. To extract substance(s) via mechanical force the plant(s) can be pressed by any known mechanical press or by altering the ambient pressure. Depending on the plant(s) and the substance(s) to be extracted even a simple filtering can be used alone or in addition to solvent(s) or mechanical force as some plants, e.g. after cutting, liberate substances, e.g. in form of liquids. Other filtering means can be used in combination with mechanical vibration, e.g. to separate solid substances such as pollen, from a plant.

The extracting step can be performed using components of a single plant or of a blend of plants. Also, one or more specific parts of plants can be used.

The at least partially fibrous residue can be mixed with the fibrous part of at least one further plant prior to preparing the sheet. In this way substances from different origin and with different properties, e.g. mechanical or pharmaceutical, can be mixed together to obtain desired product properties. Also, the at least partially fibrous residue can be mixed with a stabilizer prior to preparing the sheet. For example, the fibrous residue can be mixed with synthetic fibers and/or natural fibers to obtain certain mechanical properties, wherein the fibers are preferably non soluble and/or are approved by food, medicinal and/or cosmetic laws.

The plant extract of step b) or e) can be mixed with a plant extract of at least one further plant prior to applying the plant extract to the sheet. Also, the plant extract of step b) or e) can be mixed with a texturing agent prior to applying the plant extract to the sheet. Texturing agents, e.g., emulsifiers or stabilizers or phosphates or dough conditioners, can be used to add or modify the overall texture, color, or surface of products.

The sheet may also comprise an additive, such as a wet strength agent, a dry strength agent, or a softening agent, so as to develop or give the sheet new properties. These properties may include, for instance, chemical, optical, sensory, or mechanical properties such as dry strength, wet strength, and/or folding resistance.

The method may further comprise the step of adding ingredients or removing ingredients, e.g. undesired compounds or impurities, from the plant extract prior to applying the plant extract of step b) or step e) to the sheet of step d). Similarly, the method may further comprise the step of adding or removing ingredients from the at least partially fibrous residue prior to applying the plant extract of step b) or step e) to the sheet of step d).

The sheet may also be used with a second sheet to form a hybrid sheet. This second sheet may be manufactured via processes known to those skilled in the art. In one embodiment, the hybrid paper may comprise the sheet according to the invention complexed with the second sheet, for instance, via a hydroentanglement process.

Other features and aspects of the present disclosure are discussed in greater detail below.

### Definitions

As used herein, a “reconstituted plant material” refers to a material formed by a process in which a plant feed stock, such as cannabis components, is extracted with a solvent to form an extract of solubles, such as water solubles, and an extracted insoluble portion or residue comprising fibrous material. The extracted and insoluble fibrous material is then formed into a sheet through any suitable process and the extract may either be discarded or reapplied to the formed sheet. The extract can be fed through various processes for concentrating the extract and optionally removing or adding various components prior to being recombined with the fibrous material. In the present disclosure, the reconstituted plant material is formed from extracted cannabis fibers optionally combined with web building fibers, such as cellulose fibers. The extract of solubles obtained from the cannabis fibers is optionally reapplied to the sheet or material.

As used herein, “extracted cannabis fibers” refers to cannabis fibers that have been subjected to an extraction process in which the cannabis has been contacted with an aqueous solution to remove water soluble components contained in the cannabis. The extraction process is different from a delignification process and from a bleaching treatment.

As used herein, “delignified” cellulosic fibers refers to fibers that have been subjected to a pulping or delignification process by which the cellulose fibers are separated from the plant material through chemical means, mechanical means, or through a combination of chemical and mechanical means.

As used herein, the term “refine” is used to mean that the plant material is subjected to a mechanical treatment that modifies the fibers of the material so that they are better suited to forming a fibrous sheet or substrate. Refining can be accomplished using a conical refiner, disks refiner, or a beater such as a Valley beater. The mechanical process exerts an abrasive and bruising action on the plant material such that the plant material is deformed and declustered. Refining is a different process than delignification and pulping.

As used herein, the term “stalk” is used to refer to the main structural portion of a plant that remains after the leaves have been removed.

As used herein, the term “hurd” is used herein to refer to the structural portion of a plant, e.g. stem) connecting the leaves or laminae to the stalk and also to the veins or ribs that extend through the leaves. The term “hurd” does not encompass the term “stalk” and vice versus.

As used herein, “cannabis” may refer to any variety of the Cannabis plant, such as Cannabis sativa or Cannabis indica or Cannabis ruderalis, for instance. More particularly, the present disclosure may refer to leaves, stems, seeds, seed shells, and flowers, or any other part of the Cannabis plant, as cannabis. Nonetheless, cannabis, as referred to herein, includes cannabis that contains average or high levels of THC and/or CBD (usually known as marijuana), hemp, which may contain low, or very low, levels of THC, industrial hemp, which may refer to a cannabis plant that contains less than 0.3% THC, or combinations thereof.

The Freeness value (° SR) measures generally the rate at which a dilute suspension of refined fibers may be drained. The freeness is measured by the Schopper Riegler Method for drainability. As used herein, freeness is measured according to Test NORM EN ISO 5267-1.

### BRIEF DESCRIPTION OF THE DRAWINGS

A full and enabling disclosure of the present disclosure is set forth more particularly in the remainder of the specification, including reference to the accompanying figures, in which:

FIG. 1a is a schematic cross sectional view of one exemplary product of the invention;

FIG. 1b is a schematic cross sectional view of an exemplary multilayer product of the invention;

FIG. 2 is a schematic plan view of a patch of the invention;

FIG. 3 is a plan view of one embodiment of a facial mask made in accordance with the present disclosure.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to a medicinal, cosmetic or dermatologic product made from extracted plant fibers and optionally treated with a plant extract. In accordance with the present disclosure, at least a portion of the extracted plant fibers are obtained from cannabis components. The cannabis components, for instance, can be subjected to an extraction process for removing water soluble components. The water insoluble components, which comprise fibers and other materials, can then be formed into a reconstituted cannabis material. The reconstituted cannabis material can be in the form of a sheet-like layer. It was discovered that the reconstituted cannabis material is well suited for being used to make a medicinal, cosmetic or dermatologic product that is designed to contact the skin of a user. For example, the reconstituted cannabis material can be used to deliver a personal care composition to the skin that can provide wellness benefits or healing benefits. The personal care composition, for instance, can contain at least one plant extract. Examples of products that can be made in accordance with the present disclosure include wound care dressings, cosmetic facial masks, and the like.

Cannabis, or the cannabis plant, may refer to both marijuana, which is generally used for medical or recreational purposes, and hemp, which is generally used in industrial applications. Cannabis is a green and/or brown mixture of dried, shredded leaves, stems, seeds, seed shells, and flowers of the plant, and may reference leaves, stems, seeds, seed shells, and flowers from a Cannabis plant, varieties of which include Cannabis sativa or Cannabis indica or Cannabis ruderalis or a hybrid. Hemp (and particularly the industrial hemp variety), have a very similar appearance to marijuana, but unlike the cannabis plant variety referred to by marijuana, hemp generally only contains low amounts of tetrahydrocannabinol (THC), where both hemp and marijuana can include high amounts of cannabidiol (CBD). For instance, hemp, and particularly industrial hemp may contain less than about 0.3% THC where the cannabis variety referred to by marijuana may contain anywhere from 5% to 30% THC.

Cannabis, for instance, is becoming a more and more popular drug for pain relief in lieu of conventional pain relief medicines, such as opioids. Opioids are powerful pain relief medications that relieve pain by acting on the nervous system. They are typically used to treat severe pain after surgery and are also used to treat chronic pain. Unfortunately, however, opioids come with many risks. For example, opioids are highly addictive which has led to an epidemic of drug misuse.

In view of the above drawbacks, more and more people in the medical community have begun to view cannabis as a legitimate alternative to the use of opioids for pain relief. Cannabis, for instance, contains two different drugs that can help relieve pain, nausea, and other symptoms. Cannabis contains, for instance, tetrahydrocannabinol (THC). THC acts on specific receptors in the brain which lead to a feeling of euphoria and a relaxed state. The highest concentrations of THC in cannabis are found in the dried flowers or buds. Cannabis is typically regulated based upon the amount of THC found in the material.

In addition to THC, cannabis also can contain cannabidiol (CBD). Although CBD does interact with pain receptors in the brain, CBD does not create the same euphoric feeling caused by THC. CBD, however, exerts pain-relieving and anti-inflammatory effects. Cannabis, and especially CBD, does not have the same addictive effect as many opioids.

Conventionally, cannabis has been used as a smoking product or incorporated into a food product and ingested. The present inventors discovered, however, that cannabis is well suited to being used in medicinal, cosmetic and dermatologic products for contact with the skin. The cannabis product, for instance, can deliver to a user not only one or more cannabinoids, but can also be used as a delivery agent for delivering to the skin various other health aids whether used for healing, skin ailment prevention, or general skin wellness.

As described above, the medicinal, cosmetic or dermatologic product of the present disclosure is formed from a reconstituted cannabis material. The reconstituted plant material of the present disclosure is generally formed from extracted cannabis components or fibers optionally combined with web building fibers. Cannabis is intended to cover all varieties of the Cannabis sativa plant species, whether or not the plant species contains detectable levels of THC. Many species of cannabis, for instance, contain THC. There are, however, cannabis plant strains that are particularly low in THC and typically referred to as industrial hemp. Industrial hemp, for instance, can contain THC in an amount less than about 1% by weight, such as in an amount less than about 0.5% by weight, such as in an amount less than about 0.3% by weight, such as in an amount less than about 0.2% by weight, such as in an amount less than about 0.1% by weight. Cannabis components used to produce a drug for medicinal or recreational use, on the other hand, can contain anywhere from 3% to over 20% by weight THC. Cannabis can be obtained from the plant Cannabis indica, Cannabis sativa, Cannabis ruderalis, or a hybrid.

In one embodiment, cannabis plant materials, such as leaves, hurds, buds, flowers, seeds, seed shells, cannabis residues, by-products of extraction, and mixtures thereof are optionally sized or ground and then subjected to an extraction process for removing water soluble components. The extracted cannabis can then be combined with web building fibers and formed into a substrate, such as a reconstituted sheet. The substrate can optionally be treated with the extract obtained from the cannabis. Alternatively, the extract obtained from the cannabis can be discarded and not recombined with the water insoluble fibers and other materials.

In forming the reconstituted plant material of the present disclosure, the cannabis components are first collected and optionally reduced in size. The cannabis components can include leaves, hurds, buds, flowers and optionally stalk components. In one embodiment, the cannabis components are obtained from cannabis plants that have a relatively low THC content. For instance, the amount of THC in the cannabis components can be less than about 1% by weight THC, such as less than about 0.3% by weight THC, such as less than about 0.2% by weight THC, such as less than about 0.1% by weight THC.

After the cannabis components are collected, the cannabis components can be subjected to a grinding operation, milling operation, or beating operation that can reduce the size of the cannabis components and/or reduce the cannabis into individual fibers.

After the cannabis is optionally reduced in size, the cannabis is subjected to an extraction process for removing water soluble components. The extraction process can provide various different benefits. For instance, the extraction process can remove constituents contained in the cannabis that are irritants. In this manner, the extraction process can dramatically reduce from the material various harsh components. In addition, subjecting the cannabis to an extraction process also cleans the plant material and removes any herbicides, pesticides, and/or microorganisms that may be present on the material.

During the extraction process, the cannabis is contacted with a solvent in order to remove the water soluble components. In one embodiment, the solvent comprises only water. In an alternative embodiment, various solvents that are water-miscible, such as alcohols (e.g., ethanol), can be combined with water to form an aqueous solvent. The water content of the aqueous solvent can, in some instances, be greater than 50 wt. % of the solvent, and particularly greater than 90 wt. % of the solvent. Deionized water, distilled water, or tap water may be employed. The amount of the solvent in the suspension can vary widely, but is generally added in an amount from about 50 wt. % to about 99 wt. %, in some embodiments from about 60 wt. % to about 95 wt. %, and in some embodiments, from about 75 wt. % to about 90 wt. % of the suspension. However, the amount of solvent can vary with the nature of the solvent, the temperature at which the extraction is to be carried out, and the type of cannabis furnish.

In addition to aqueous solvents, non-aqueous solvents can also be used. The solvent, for instance, can be an oil or a fat, isopropyl alcohol, butane, or supercritical CO₂. In one embodiment, a multi-phase solvent can be used that includes a combination of water with an oil or fat.

After forming the solvent/cannabis furnish mixture, some or all of a soluble fraction of the furnish mixture may be separated from the mixture. The aqueous solvent/cannabis furnish mixture can be agitated by stirring, shaking or otherwise mixing the mixture in order to increase the rate of solubilization. Typically, the process is carried out for about one-half hour to about 6 hours. Process temperatures may range from about 10° C. to about 100° C., such as from about 40° C. to about 90° C.

After the cannabis materials are soaked in an extractant, the insoluble cannabis material can be mechanically separated from the cannabis liquor or extract using a press. Once the soluble fraction is separated from the cannabis furnish or insoluble fraction, the soluble fraction can be discarded or further processed, such as by being concentrated. The soluble fraction can be concentrated using any known type of concentrator, such as a vacuum evaporator. In one embodiment of the present disclosure, the soluble fraction can be highly concentrated. In one embodiment, for instance, the cannabis soluble fraction can be evaporated so as to have a final brix of from about 10% to about 50%, such as from about 15% to about 35%.

The resulting concentrated cannabis soluble fraction may be used in a separate process, or can be later coated onto the reconstituted plant material of the present disclosure as will be described in greater detail below.

The resulting water insoluble cannabis fraction is generally in an unrefined state. The cannabis material can comprise particles and fibers. In one embodiment, the insoluble and extracted cannabis fraction can be subjected to a refining process. For instance, the extracted cannabis material can be fed through any suitable refining device, such as a conical refiner or a disk refiner. Other refining devices that may be used include a beater, such as a Valley beater. Refining can occur while the cannabis materials are moist or after being combined with water. For instance, in one embodiment, refining can occur while the cannabis material is at a consistency of less than about 10%, such as less than about 5%, such as less than about 3%.

In accordance with the present disclosure, the extracted cannabis material can then be combined with web building fibers to form a fiber substrate, such as a reconstituted plant material. For example, the extracted cannabis can be combined with water or an aqueous solution to form a slurry. The web building fibers, such as delignified cellulosic fibers, can be combined with the cannabis material in forming the slurry. The fiber slurry is then used to form a continuous reconstituted sheet.

The fibrous slurry containing the extracted cannabis material and optionally the web building fibers can be subjected to a refining process. Alternatively, the extracted cannabis material can be fed through a refining process and then combined with the web building fibers. In still another aspect, the extracted cannabis material can be fed through a refining process, combined with the web building fibers, and the resulting mixture can then be fed through a further refining process.

The amount the extracted cannabis material and/or the web building fibers are refined can impact various properties of the reconstituted material formed later. For instance, increasing the amount the cannabis material and/or the web building fibers are refined can make it easier to cut the layer of the reconstituted material. In addition, increasing the amount of refining can also improve the tactile properties of the sheet-like layer.

In one aspect, the extracted cannabis material and the web building fibers together can have a refining level or refining degree of greater than about 40° SR, such as greater than about 60° SR, such as greater than about 65° SR, such as greater than about 70° SR, such as greater than about 73° SR. The refining level can generally be less than about 100° SR, such as less than about 90° SR, such as less than about 80° SR.

In one embodiment, the fiber slurry is fed to a papermaking process that can include a forming wire, gravity drain, suction drain, a felt press, and a dryer, such as a Yankee dryer, a drum dryer, or the like. For example, in one embodiment, the fiber slurry is formed into a continuous sheet on a Fourdrinier table. One advantage to combining the extracted cannabis with the cellulosic fibers is that the resulting fiber furnish can be processed on conventional papermaking equipment.

In one embodiment, the fiber slurry is laid onto a porous forming surface and formed into a sheet. Excess water is removed by a gravity drain and/or a suction drain. In addition, various presses can be used to facilitate water removal. The formed sheet can be dried and further treated.

Reconstituted substrates can also be made using various other different methods. For example, in one embodiment, the extracted cannabis and web building fibers may be extruded into a reconstituted material. In one embodiment, the reconstituted material can also be subjected to an expansion process. Expanded sheets can be made using, for instance, a gas, such as carbon dioxide, or by using a foaming agent. Suitable expansion mediums include starch, pullulan or other polysaccharides, solid foaming agents, inorganic salts and organic acids that provide in situ gaseous components, organic gaseous agents, inorganic gaseous agents, and volatile liquid foaming agents. Extruding also allows for the formation of rods or strands in addition to sheet materials.

Optionally, the reconstituted plant material that is produced can also be treated with the cannabis soluble portion, such as a concentrated cannabis soluble portion that was separated from the insoluble fraction. The cannabis soluble portion can be applied to the web using various application methods, such as spraying, using a size press, saturating, etc. The amount of water soluble cannabis extracts applied to the reconstituted material can depend upon various factors and the anticipated end use application.

In one embodiment, the amount of water soluble cannabis extracts contained in the reconstituted material can be minimal. For instance, the reconstituted material can contain water soluble cannabis extracts in an amount of about 10% by weight or less, such as in an amount less than about 8% by weight, such as in an amount less than about 6% by weight, such as in an amount less than about 4% by weight.

In one embodiment, greater amounts of water soluble cannabis extracts may be applied to the reconstituted material. For example, in an alternative embodiment, the water soluble cannabis extracts can be applied to the reconstituted material such that the reconstituted material contains the water soluble cannabis extracts in an amount greater than about 8% by weight, such as in an amount greater than about 10% by weight, such as in an amount greater than about 12% by weight, and generally in an amount less than about 60% by weight, such as in an amount less than about 30% by weight, such as in an amount less than about 25% by weight, such as in an amount less than about 23% by weight, such as in an amount less than about 21% by weight, such as in an amount less than about 20% by weight, such as in an amount less than about 18% by weight.

As described above, the reconstituted cannabis material of the present disclosure can be produced from various parts of the cannabis plant, including the hurds, leaves, buds, and flowers. These different parts of the plant can be combined in different ratios and amounts depending upon the particular application and the desired result. Although the reconstituted cannabis material can be made exclusively from cannabis leaves and hurds or can be made exclusively from cannabis buds and flowers, in one embodiment, the reconstituted material is made from a mixture of leaves and hurds combined with buds and/or flowers. For example, in one embodiment, the weight ratio between the leaves and hurds and the buds and/or flowers is from about 1:8 to about 8:1, such as from about 1:5 to about 5:1, such as from about 1:4 to about 4:1, such as from about 1:2 to about 2:1. In one embodiment, the ratio can be about 1:1.

In one embodiment, the reconstituted cannabis material may contain cannabis leaves and hurds in an amount greater than about 10% by weight, such as in an amount greater than about 20% by weight, such as in an amount greater than about 30% by weight, and generally in an amount less than about 70% by weight, such as in an amount less than about 60% by weight, such as in an amount less than about 50% by weight, such as in an amount less than about 40% by weight. Similarly, the reconstituted cannabis material may contain buds and/or flowers in an amount greater than about 10% by weight, such as in an amount greater than about 20% by weight, such as in an amount greater than about 30% by weight, such as in an amount greater than about 40% by weight, such as in an amount greater than about 50% by weight, such as in an amount greater than about 60% by weight, and generally in an amount less than about 80% by weight, such as in an amount less than about 70% by weight, such as in an amount less than about 60% by weight, such as in an amount less than about 50% by weight.

The cannabis material can be combined with web building fibers. The web building fibers are incorporated into the reconstituted plant material or fiber substrate in an amount sufficient to provide strength and integrity to the resulting material. Web building fibers can also be incorporated into the reconstituted plant material so as to trap and prevent cannabis fibers and other cannabis components from separating from the fiber substrate. In general, any suitable web building fibers can be incorporated into the reconstituted plant material in order to improve one or more physical properties of the reconstituted material.

Various different types of web building fibers may be used. In one embodiment, the web building fibers are delignified cellulosic fibers. For instance, the web building fibers may comprise wood pulp fibers such as softwood fibers or hardwood fibers. Other cellulosic fibers that may be used include flax fibers, hemp fibers, abaca fibers, bamboo fibers, coconut fibers, cotton fibers, kapok fibers, ramie fibers, jute fibers, or mixtures thereof. In one particular embodiment, the reconstituted plant material contains softwood fibers alone or in combination with other fibers such as hardwood fibers, hemp fibers, or the like.

In general, the web building fibers are present in the reconstituted plant material in an amount greater than about 3% by weight, such as in an amount greater than about 10% by weight, such as in an amount greater than about 15% by weight, such as in an amount greater than about 20% by weight, such as in an amount greater than about 25% by weight, such as in an amount greater than about 30% by weight, such as in an amount greater than about 35% by weight, such as in an amount greater than about 40% by weight. The web building fibers are generally present in the reconstituted plant material in an amount less than about 60% by weight, such as in an amount less than about 55% by weight, such as in an amount less than about 50% by weight, such as in an amount less than about 45% by weight.

In one aspect, wood pulp fibers may be combined with the extracted cannabis material. When using wood pulp fibers as the web building fibers, the wood pulp fibers can be present in an amount greater than about 15% by weight, such as in an amount greater than about 18% by weight, and generally in an amount less than about 30% by weight, such as in an amount less than about 28% by weight.

Alternatively, the extracted cannabis material can be combined with hemp pulp fibers. The hemp fibers can be present in an amount generally greater than about 15% by weight, such as greater than about 25% by weight, such as greater than about 35% by weight, and generally less than about 55% by weight, such as less than about 50% by weight, such as less than about 45% by weight.

In one embodiment, the web building fibers incorporated into the reconstituted plant material include a combination of longer fibers and shorter fibers. The longer fibers can generally have an average length of greater than about 2 mm, while the shorter fibers can generally have an average length of less than about 1.5 mm. The longer fibers can be used to improve strength and integrity, while the shorter fibers can better retain the cannabis fibers and other components within the fiber substrate. In one embodiment, for instance, the short fibers may be present in the reconstituted plant material in an amount greater than about 3% by weight, such as in an amount greater than about 5% by weight, and generally in an amount less than about 20% by weight. The longer fibers, on the other hand, can be present in the reconstituted web material in an amount greater than about 2% by weight, such as in an amount greater than about 5% by weight, and generally in an amount less than about 30% by weight, such as in an amount less than about 15% by weight. In one embodiment, the shorter fibers comprise hardwood fibers, while the longer fibers comprise softwood fibers. The weight ratio between the longer fibers and the shorter fibers, for example, can be from about 6:1 to about 1:0.75, such as from about 4:1 to about 1:1.

In addition to extracted cannabis fibers and web building fibers, the reconstituted cannabis material of the present disclosure can also contain various other plant fibers. In one embodiment, for instance, the reconstituted cannabis material can contain extracted cocoa husk fibers.

Cocoa materials for use in the present disclosure are obtained from Theobroma cacao, which is also referred to as the cacao tree. The cacao tree is in the evergreen family and is native to tropical regions. The cacao tree produces a fruit, referred to as a cacao pod. Cacao pods are generally yellow to orange in color and can weigh over one pound when ripe. The pod contains anywhere from 10 to about 80 cocoa beans that are used to produce chocolate, juices, jelly, and the like. After the beans are removed from the cacao pod, the cocoa beans are dried and cured or fermented by being exposed to sunlight and/or ultraviolet light. Each individual bean is covered in a husk or shell. The husk or shell is removed from the bean prior to using the bean for producing food products. The reconstituted plant material of the present disclosure can contain the cocoa shells or husks, although other components of the cacao pod may also be used.

The cocoa shell or husk contains fibers which are well suited to producing substrates and web materials. In one embodiment, the cocoa husks are optionally sized or ground and then subjected to an extraction process for removing water soluble components. The extraction process can occur separate from the cannabis materials or the cocoa husk materials can be combined with the raw cannabis materials and subjected to the same extraction process together.

The amount of extracted cocoa husk fibers contained in the reconstituted plant material can depend upon the particular application and the desired result. Extracted cocoa husk fibers, for instance, can be present in the reconstituted plant material in an amount anywhere from 1% by weight to about 80% by weight, such as from about 5% by weight to about 50% by weight. For example, the extracted cocoa husk fibers can be present in the reconstituted plant material in an amount greater than about 10% by weight, such as in an amount greater than about 20% by weight, such as in an amount greater than about 30% by weight, and generally in an amount less than about 60% by weight, such as in an amount less than about 50% by weight, such as in an amount less than about 40% by weight.

In one embodiment, the reconstituted web material can further contain a humectant. The humectant can be incorporated into the reconstituted plant material for various different reasons in order to provide different benefits and advantages. For instance, in one embodiment, a humectant may be incorporated into the reconstituted plant material in order to improve the processability and handling of the resulting fiber substrate.

Various different humectants can be incorporated into the reconstituted plant material. The humectant, for instance, may comprise glycerol, propylene glycol, or mixtures thereof. Other humectants that may be used include sorbitol, triethylene glycol, lactic acid, glyceryl diacetate, glyceryl triacetate, triethyl citrate, isopropyl myristate, and mixtures thereof, including mixtures with glycerol and/or propylene glycol.

As described above, the amount of humectant applied to the reconstituted plant material can depend upon various factors. In one embodiment, for instance, the humectant is present on the reconstituted plant material in an amount less than about 25% by weight, such as in an amount less than about 20% by weight, and generally in an amount less than about 15% by weight. The humectant may be present on the plant material in an amount greater than about 3% by weight, such as in an amount greater than about 5% by weight.

The reconstituted plant material of the present disclosure may also optionally contain a filler. The filler can comprise particles incorporated into the reconstituted web material for any desired purpose, such as for facilitating formation of the reconstituted plant material and/or for affecting the appearance of the material. Filler particles that may be incorporated into the reconstituted web material can be made from calcium carbonate, magnesium oxide, titanium dioxide, kaolin clay, barium sulfate, a silicate, bentonite, mica, or mixtures thereof. Filler particles can optionally be incorporated into the reconstituted web material in an amount greater than about 1% by weight, such as in an amount greater than about 5% by weight, such as in an amount greater than about 10% by weight, and generally in an amount less than about 30% by weight, such as in an amount less than about 25% by weight, such as in an amount less than about 20% by weight, such as in an amount less than about 15% by weight.

The reconstituted cannabis material of the present disclosure can also be combined with various herbal fillers. The reconstituted cannabis material, for instance, can be homogeneously blended with various different types of herbal fillers. The weight ratio between the reconstituted cannabis material and the herbal filler can generally be from about 1:8 to about 8:1, such as from about 1:5 to about 5:1, such as from about 2:1 to about 1:2.

For instance, the reconstituted plant material of the present disclosure can also be combined with fillers made from herbal plant materials, such as botanical plants, and trees, such as cocoa tree, coffee tree or coffee bean, tea tree or tea leaf, vine, ginger, ginkgo, camomile, tomato, ivy, mate, rooibos, cucumber, mint, a cereal such as wheat, barley or rye, or other trees such as broadleaved or resinous trees, and the like, as well as combinations thereof.

In general, the reconstituted plant material has a basis weight of greater than about 25 gsm, such as greater than about 40 gsm, such as greater than about 45 gsm, such as greater than about 55 gsm. The basis weight of the reconstituted plant material is generally less than about 100 gsm, such as less than about 85 gsm.

The reconstituted cannabis material of the present disclosure is well suited to receiving various different topical additives. The topical additives, for instance, may comprise one or more plant extracts. The topical additive can also comprise a health agent, such as a medicament, a cosmetic additive, or a dermatologic additive. In one embodiment, the topical additive can be for transferring to the skin of a user. For example, in certain embodiments, the product of the present disclosure may have multiple layers.

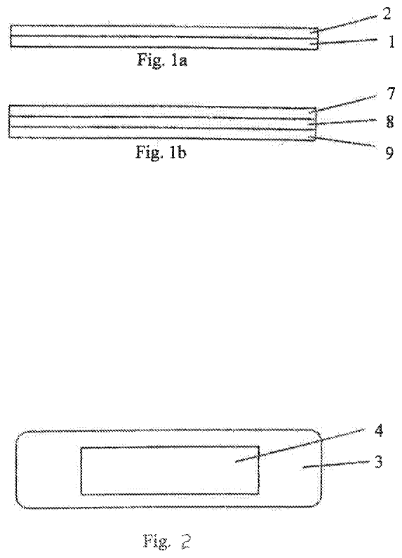

FIG. 1a shows a schematic cross sectional view of a product according to the present disclosure. The first layer 1 comprises a fibrous plant product and the second layer 2 comprises a plant extract. The first layer may have a thickness of 100 μm to 0.5 cm, preferably 0.2 mm to 5 mm. Instead of having two substantially separate layers, the plant extract can partially or entirely enter or penetrate into the fibrous plant product. The first layer 1 can have a porous structure to facilitate the plant extract entering into the fibrous plant product. Also, the fibrous plant product can be small pieces of any shape or a paste or a powder, and the plant extract can be applied to the plant product.

The product according to the invention may comprise two, three, four, five, or more layers, e.g., a first layer 1 comprising a fibrous plant product, a second layer 2 comprising a plant extract with the first substance(s), a third layer (not shown) comprising a plant extract with the second substance(s), etc. Each layer may comprise different substance(s) offering a specific effect. Also, additional layers or additional amounts of the respective substances in the existing layers can be provided to control the sequence and/or amount and/or speed of substances being released from the product.

One or both of the plant extract and the fibrous plant product may further comprise a matrix of a texturing agent, such as a non-crosslinked hydrocolloid polymer of natural or synthetic origin, preferably of natural origin. The texturing agent can be selected from at least one of:
natural agents of plant origin such as carob gum, guar gum, pectins, alginates, carrageenans, agar-agar, gum arabic, and cellulose;
natural agents of microbial origin such as xanthan gum natural agents, gellan gum, hyaluronic acid, and dextran;
natural agents of animal origin, such as gelatin, collagen, and chitosan; and/or
mineral agents, such as clays and silicas, and synthetic polymers, such as polyacrylic and polyacrylamide agents.

The fibrous plant product may further comprise an additive that can develop or give the product new properties, for instance chemical, optical, sensory, or mechanical properties such as dry strength, wet strength, and/or folding resistance. The additive may be chosen from a wet strength agent, a dry strength agent, a softening agent, a lotion composition, a wetting agent, latex, a cannabinoid such as CBD and THC, and mixtures thereof; particularly chosen from a wet strength agent, a dry strength agent, a lotion composition, a cannabinoid, and mixtures thereof; and most particularly a wet strength agent.

Typically, the amount of additive is less that 3% by weight of dry matter of the plant product, in particular from 0.5% to 2% by weight of dry matter of the plant product, and most particularly from 1.3% to 1.7% by weight of dry matter of the plant product.

A wet strength agent makes it possible to reduce the potential degradation of the plant product is placed in contact with a liquid, such as water. Typically, the wet strength agent may be chosen from polyamides, such as an epichlorohydrin resin, a polyamine-epichlorohydrin resin, a polyamide-epichlorohydrin resin, a poly(aminoamide)-epichlorohydrin resin, a urea formaldehyde resin, a melamine-formaldehyde resin, an alkyl-ketene dimer, alkylsuccinic anhydride, a polyvinylamine, an oxidized polysaccharide, and mixtures thereof.

A dry strength agent makes it possible to increase the strength of the plant product if the latter is subjected to substantial mechanical stresses. The dry strength agent may be chosen from starches and modified gums, cellulose polymers, or synthetic polymers, for instance carboxymethylcellulose, polyacrylamides, and mixtures thereof.

A softening agent makes it possible to improve the softness of the plant product according to the invention. Typically, a softening agent is a fatty acid, a siloxane compound, a silicone compound, an aminosilicone compound, an Aloe vera extract, a sweet almond extract, a chamomile extract, a quaternary ammonium compound, and mixtures thereof.

The sheet formed by the fibrous plant product may also be used with a second sheet to form a hybrid sheet. This second sheet may be manufactured via processes known to those skilled in the art, such as the wet laid process; the dry laid carded process using long artificial fibers, long natural fibers, cellulose fibers, and mixtures thereof; or the airlaid process using long artificial fibers, long natural fibers, cellulose fibers, and mixtures thereof. This second sheet may also be a spunbound support.

The plant product sheet and second sheet may be complexed via additional treatments allowing manufacture of a multilayer hybrid sheet, such as hydroentanglement. Hydroentanglement uses high-pressure water jets to entangle the fibers. The sheet(s) is gripped between a grate and a compression strip and is first compacted and moistened to prevent the formation of air pockets. The sheet(s), while circulating on a perforated cylinder covered with a fine gauze, is subjected to high-pressure water jets on one face, then on the other. Typically, the water jets are emitted by injectors, i.e. holes, typically from 80 μm to 150 μm in diameter, arranged at a rate of 1 to 3 holes per mm, on rows typically 3 mm to 5 mm apart. The water pressure increases from the first to last injectors. To avoid inundating the sheet(s), the interior of the cylinders is under negative pressure. The residual water is removed first by suction, then by drying.

The sensory properties of the sheet(s) that have undergone a hydroentanglement treatment are improved, in particular softness and absorption capacity. Furthermore, said sheet(s) have greater tensile strength and is easy to shape.

The invention can be used in many areas, such as for medicinal or cosmetic or dermatologic use or any combination of these areas.

The product of the invention provides improved properties as regards at least one of substance concentration and substance release. Commonly known ways of administering a plant product can be improved and current difficult ways for administering a plant product can be used more effectively.

The reason for this improvement is that, due to the processing of the raw material(s) according to the invention, a controlled amount of selected substances can be placed on the product, i.e. in the fibrous plant product or the plant extract. If desired, a cannabis plant can substantially be reconstituted or reconstructed so that the final product comprises many or substantially all substances of the raw material. The reconstructed product is advantageous in comparison to the original plant, as the substances from the reconstructed product can be released in a controlled way, e.g., faster than from the natural plant. In addition, it may be desired to mix other substances from other plants or synthetical substances into the product to alter its mechanical or medicinal/dermatologic/cosmetic properties. Likewise it can be desired to separate certain undesired substances, e.g., pesticides, metals, or polyphenols.

The product according to the invention can also be designed to comprise different substances for different effects. In particular, the product can be designed to release different substances at different times and rates. In consequence, it is possible that a first substance provides a first effect and afterwards a second substance provides another effect. The times when the substances provide an effect can at least partially overlap. For example, it is possible to provide a dermatologic path which first provides a cooling effect and, subsequently or overlapping, releases a substance with a displeasing side effect such as burning or pricking.

### Medicinal Applications

According to one aspect, the invention can be used in medicinal applications. In particular, due to the improved properties of the product according to the invention, the administering of medicinal substances becomes more efficient.

When used in medicinal applications, the product of the present disclosure can be used as a wound dressing or can be used as a sublingual strip. Having at least one of a higher concentration of the substance(s), a higher liberation rate of the substance(s), and better solubility properties makes wound or sublingual administration of a plant product possible.

When designing a product for medicinal purposes, the reconstituted cannabis material can be blended with various other plants if desired. Plants for medicinal applications can be selected from one or more of Achillea millefolium L.; Adhatoda vasica Nees; Aesculus hippocastanum L.; Agrimonia eupatoria L.; Agropyron repens (L.); Agropyron repens (L.) P. Beauv.; Allium sativum L.; Allium cepa L.; Aloe barbadensis Miller; Aloe ferox Miller; Althaea officinalis L.; Andrographis paniculata Nees; Angelica sinensis (Oliv.) Diels; Arctium lappa L.; Arctostaphylos uva-ursi (L.) Sprang.; Arnica montana L.; Artemisia absinthium L.; Avena sativa L.; Betula pendula Roth Betula pubescens Ehrh.; Calendula officinalis L.; Camellia sinensis (L.) Kuntze; Capsella bursa-pastoris (L.) Medikus; Capsicum annuum L. Heiser; Carum carvi L.; Cassia senna L.; Cassia angustifolia Vahl; Centaurium erythraea Rafn.; Centella asiatica L. Urban; Cetraria islandica (L.) Acharius s.I., Chamaemelum nobile (L.) syn. Anthemis nobilis L.; Chamaemelum nobile (L.); Anthemis nobilis L.; Chamomilla recutita (L.) Rauschert; Matricaria recutita (L.); Chelidonium majus L.; Cichorium intybus L.; Cimicifuga racemosa (L.) Nutt.; Cinnamomum verum J. S. Presl, Cinnamomum zeylanicum Nees; Citrus bergamia Risso; Citrus bergamia Risso & Poiteau.; Citrus spp.; Cola nitida (Vent.) Cola acuminata (P. Beauv.); Cola acuminata (P. Beauv.) Schott et Endl.; Commiphora molmol Engler; Crataegus monogyna Jacq. (Lindm.); Crataegus laevigata (Poir.) DC; Cucurbita pepo L.; Curcuma longa L.; Cynara scolymus L.; Curcuma xanthorrhiza Roxb.; C. xanthorrhiza D. Dietrich.; Echinacea angustifolia DC.; Echinacea pallida (Nutt.) Nutt.; Echinacea purpurea (L.) Moench.; Eleutherococcus senticosus (Rupr. et Maxim.) Maxim.; Equisetum arvense L.; Erysimum officinale L.; Eschscholtzia california Cham.; Eucalyptus globulus Labill.; Eucalyptus polybractea R. T. Baker; Eucalyptus smithii R. T. Baker.; Euphrasia officinalis L.; Filipendula ulmaria (L.) Maxim.; Spiraea ulmaria L.; Foeniculum vulgare Miller subsp. vulgare var. vulgare; Fragaria vesca L.; Fraxinus excelsior L.; Fucus vesiculus L.; Fumaria officinalis L.; Gentiana lutea L.; Ginkgo biloba L.; Glycyrrhiza glabra L.; Glycyrrhiza inflata Bat.; Glycyrrhiza uralensis Fisch.; Grindelia robusta Nutt.; Grindelia squarrosa (Pursh) Dunal, Grindelia humilis Hook. et Arn., Grindel; Lavandula angustifolia Mill.; Lavendula officinalis Chaix; Leonurus cardiaca L.; Levisticum officinale Koch.; Linum usitatissimum L.; Marrubium vulgare L.; Matricaria recutita L.; Melaleuca alternifolia (Maiden and Betche) Cheel; Melilotus officinalis (L.) Lam.; Melissa officinalis L.; Mentha x piperita L.; Oenothera biennis L.; Oenothera lamarckiana L.; Olea europaea L.; Ononis spinosa L.; Ononis arvensis L.; Origanum dictamnus L.; Orthosiphon stamineus; Orthosiphon stamineus Benth.; Panax ginseng C. A. Meyer.; Passiflora incarnata L.; Paullinia cupana Kunth; Pelargonium sidoides DC; Pelargonium reniforme Curt.; Peumus boldus Molina; Phaseolus vulgaris L.; Picrorhiza kurroa Royle ex. Benth.; Pimpinella anisum L.; Plantago lanceolata L.; Plantago ovata Forssk.; Plantago afra L.; Plantago indica L.; Polypodium vulgare L.; Potentilla erecta (L.) Raeusch.; Primula veris L.; Primula elatior (L.) Hill; Prunus africana (Hook f.) Kalkm.; Quercus robur L.; Quercus petraea (Matt.) Liebl.; Quercus pubescens Wind.; Rhamnus purshianus D. C.; Rhamnus frangula L.; Rheum palmatum L.; Rheum officinale Baillon; Rhodiola rosea L.; Ribes nigrum L.; Rosa centifolia L.; Rosa gallica L.; Rosa damascena Mill.; Rosmarinus officinalis L.; Rubus idaeus L.; Ruscus aculeatus L.; Salix [various species including S. purpurea L.; S. daphnoides Vill.; S. fragilis L.]; Salvia officinalis L.; Sambucus nigra L.; Serenoa repens (Bartram) Small; Sabal serrulata (Michaux) Nichols; Silybum marianum L. Gaertner; Solanum dulcamara L.; Solidago virgaurea L.; Symphytum officinale L.; Syzygium aromaticum (L.); Syzygium aromaticum (L.) Merill et L. M. Perry; Tanacetum parthenium (L.) Schultz Bip.; Taraxacum officinale Weber ex Wigg.; Thymus vulgaris L.; Thymus zygis Loefl. ex L.; Tilia cordata Miller; Tilia platyphyllos Scop.; Tilia x vulgaris Heyne Tilia tomentosa Moench; Trigonella foenum-graecum L.; Uncariae tomentosae (Willd.) DC.; Urtica dioica L.; Urtica urens L.; Vaccinium myrtillus L.; Valeriana officinalis L.; Verbascum thapsus L.; Verbascum densiflorum Bertol.; V. thapsiforme Schrad; Verbascum phlomoides L.; Viola tricolor L.; Viscum album L.; Vitex agnus-castus L.; Vitis vinifera L.; and Zingiber officinalis L.

### Dermatologic Applications

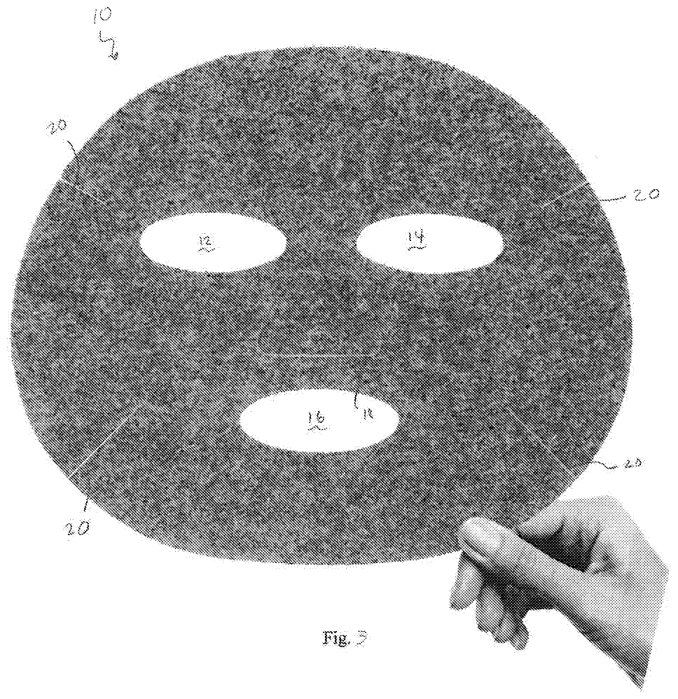

The product of the invention can also be comprised of a dermatological product. For example, a patch as illustrated in FIG. 2 may comprise an adhesive area 3 and a pharmaceutically active area 4 comprising the product according to the invention. The patch can be applied to skin to be treated, wherein the pharmaceutically active area 4 faces towards the skin to be treated. Once the patch is applied by pressing it against the skin to be treated, the adhesive area 3 secures the patch to the skin and the pharmaceutically active area 4 can unfold its effect. The patch can have various shapes.

Referring to FIG. 3, in addition to a patch or wound dressing, the product of the present disclosure can be in the form of a face mask 10. The face mask 10 can be formed from the reconstituted cannabis material as described above. The face mask 10 can be a single layer product or can be a multilayer product, such as shown in FIG. 1a or 16. The mask 10 can have a preformed shape corresponding to a human face. As shown in FIG. 3, the mask 10 can include eye openings 12 and 14 and a mouth opening 16. In addition, the mask 10 can include one or more score lines 18 that later form an opening for the nose. In addition, the mask 10 can include score lines 20 for allowing better form-fitting properties when applied to a user's face, such that, for instance, the sides of the mask may touch the user's face. The mask 10 may include adhesive or may be adhesive-free. Also, the mask may be rehydrated prior to use, e.g., by applying cold or warm water or other liquids or solvents or mixture of them. In this way, release of pharmaceutically active substances can be improved.

One or both of the plant extract and the fibrous plant product, in particular the substances contained therein, are able to act on the skin, for example by diffusion or penetration into the skin, or simply by the effect of surface contact with skin. One or more of the following substances can be used: chamomile, wild pansy, aloe vera, tea tree, St. John's Wort, burdock, witch hazel, willow, dandelion, or oregano.

An exemplary multilayer facial mask is illustrated in FIG. 1b. Layer 8 comprises reconstituted cannabis material with a fibrous plant product and a plant extract. Layer 9 is a lower layer which comes into contact with the skin and comprises cellulosic fibers, e.g. hemp, which may offer a softer surface. Layer 9 may have a weight from 10 gsm to 100 gsm. Preferably layer 9 is porous enough to let plant extract reach the skin. Layer 9 may also contain a hydrophobic or hydrophilic solution (such as water or humectants or alcohols or a blend of them) in order to facilitate plant extract diffusion. The same solution can also contain one or more ingredients such as extracts, scents, coloring agents, preserving agents, emulsifiers, lubricants, acid and/or base to adjust pH. Layer 7 is an upper (outside) layer comprising synthetic fibers, such as polyamide, polyethylene, polypropylene, rayon such as Viscose/Tencel, and polyester, as well as blends of thereof. Layer 7 may have a weight from 10 gsm to 500 gsm depending on product applications. Layer 7 may provide appropriate physical characteristics to the end product.

A mask according to the invention may comprise different zones comprising different active substances, e.g., a first substance or mix of substances for the forehead and cheek region, a second substance or mix of substances for the eye region, and a third substance or mix of substances for the nose region.

The reconstituted cannabis material of the present disclosure, as described above, can be combined with various plants. When used in dermatologic applications, for instance, one or more of the following plants may be combined with the cannabis material. The plants include Abies spp.; Achillea officinalis; Aesculus hippocastanum; Agrimonia eupatoria; Aloe spp.; Althaea officinalis; Anthemis nobilis; Arctium majus; Arnica montana; Balsamita major; Brassica spp.; Calendula officinalis; Capsella bursa pastoris; Centaurea cyanus; Centella asiatica; Cinchona spp.; Cochlearia armoracia; Commiphora spp.; Coryllus avelana; Crocus sativus; Cupressus sempervirens; Erysimum spp.; Eucalyptus spp.; Ficaria ranunculoides; Filipendula ulmaria; Fucus vesiculosus; Ginkgo biloba; Glycyrrhiza spp.; Hamamelis virginiana; Hedera helix; Hypericum perforatum, Juglans regia; Krameria tetrandra; Lamium spp.; Lavandula spp.; Lippia citriodora; Malva sylvestris; Matricaria recutita; Melaleuca spp.; Melilotus officinalis; Mentha spp.; Nuphar luteum, Origanum majorana; Paullinia cupana; Petroselinum crispum, Pinus spp.; Plantago spp.; Polygonum bistorta; Populus spp.; Potentilla erecta; Quercus spp.; Raphanus sativus; Rheum officinale; Ribes nigrum; Rosa spp.; Rubus spp.; Ruscus aculeatus; Salicaria officinalis; Salix spp.; Salvia spp.; Satureia montana; Symphytum officinale; Syzygium aromaticum; Thea sinensis; Thea spp.; Thymus spp.; Tilia spp.; Tropaeolum majus; Vaccinium myrtillus; Verbascum thapsiforme; Verbena officinalis; Viburnum spp.; Viola spp.; Vitis vinifera; and Ziziphus jujuba.

Preferred substances in the product according to the invention are selected from one or more of an antioxidant agent, an anti-radical agent, a depigmenting agent, a liporegulating agent, an anti-acne agent, an antiseborrhoeic agent, an anti-aging agent, a softener, an anti-wrinkle agent, an anti-inflammatory agent, a healing agent, a hydrating agent, an antibacterial agent, an antifungal agent, a vitamin, a protein, an amino acid, a fatty oil, an essential oil agent, a phytosterol, a ceramide, a clay, and a UV filter.

### Cosmetic Applications

The product according to the invention may also be used in cosmetic applications. A cosmetic mask may correspond to the mask as explained in the context of dermatologic applications but comprising substances with a cosmetic effect rather than substances with a medicinal and/or dermatologic effect. As already mentioned, also combinations are possible, e.g. a mask comprising at least one of a cosmetic, dermatologic, and medicinal effect.

Just like with other applications, it is also desirable in cosmetic applications to not only control the amount of desired substances, but also to remove selectively undesired substances like pesticides, metals, polyphenols, or sensitizing agents. Indeed, it has been proven that molecules like polyphenols can damage hair or skin.

According to the invention it is possible to alter the properties of the resulting product by adding further excipients like extracts, scents, coloring agents, preserving agents, emulsifiers, lubricants, or acid or base to adjust pH.

Plants for cosmetic applications that can be combined with the cannabis material can be selected from one or more of Achillea millefolium; Actinidia chinensis; Aesculus hippocastanum; Agrimonia eupatoria; Agropyrum repens; Aloe spp.; Althaea spp.; Amyris balsamifera; Ananas sativus; Anethum graveolens; Angelica archangelica; Arctium majus; Arctostaphyllos uva ursi; Arnica montana; Artemisia spp.; Bambusa arundinacea; Artocarpus heterophyllus; Ascophyllum nodosum, Asparagus officinalis; Avena sativa; Bambusa arundinacea; Bandeiraea simplicifolia; Bergenia crassifolia; Betula spp.; Boerhavia diffusa; Boswellia carted; Brassica spp.; Broussonetia papyrifera; Calendula officinalis; Calluna vulgaris; Camellia spp.; Cananga odorata; Capsicum spp.; Carapa guaianensis; Carica papaya; Carum carvi; Cassia spp.; Castanea spp.; Centaurea cyanus; Centella asiatica; Chamomilla spp.; Chenopodium quinoa; Chondrus crispus; Chrysanthellum indicum, Chrysanthemum cinerariaefolium; Cichorium intybus; Cinchona spp.; Cinnamomum spp.; Cistus labdaniferus; Citrullus spp.; Citrus spp.; Cnicus benedictus; Cochlearia officinalis; Coffea spp.; Commiphora abyssinica; Coriandrum sativum, Corylus avelana; Crithmum maritimum, Crocus spp.; Cucumis sativus; Cucurbita spp.; Cupressus sempervirens, Curculigo orchioides; Curcuma spp.; Cyathea medullaris, Cydonia vulgaris; Cymbopogon spp.; Cynara scolymus, Daucus carota Dioscorea spp.; Drosera spp.; Echinacea spp.; Eclipta prostrate; Epilobium angustifolium, Equisetum arvense; Erica cinerea; Euonymus europaeus; Euphorbia spp.; Euphrasia officinalis; Filipendula ulmaria, Foeniculum spp.; Fragaria spp.; Fraxinus spp.; Fucus spp.; Fumaria officinalis; Garcinia cambodgia, Gaultheria procumbens, Geranium robertianum, Ginkgo biloba; Glycine soja; Glycyrrhiza glabra; Gossypium sp.; Grindelia spp.; Haematoxylum campechianum, Hamamelis virginiana; Harpagophytum procumbens, Hedera helix; Helianthus annuus; Helichrysum italicum, Hibiscus sabdariffa; Hieracium pilosella; Himanthalia elongata; Humulus lupulus; Hypericum perforatum, Hyssopus officinalis; Ilex spp.; Ipomoea spp.; Iris spp.; Jasminum spp.; Juniperus spp.; Krameria triandra; Larix decidua; Laminaria spp.; Lamium spp.; Larrea divaritica; Laurus nobilis; Lavandula spp.; Lithothamnium calcareum, Lythrum salicaria; Mangifera indica; Marrubium vulgare; Marsdenia condurango; Melaleuca spp.; Melilotus officinalis; Melissa officinalis; Mentha spp.; Mucuna pruriens; Musa spp.; Myrtus communis, Myrica cerifera; Nasturtium officinalis; Nelumbo nucifera; Nephelium longana; Nicotiana spp.; Nigella sativa; Nuphar spp.; Ocimum basilicum, Olea europaea; Opuntia spp.; Orchis mascula, Origanum spp.; Oryza spp.; Palmaria palmata, Panax ginseng; Papaver rhoeas; Paullinia cupana; Persea spp.; Petroselinum spp.; Phaseolus spp.; Pimenta spp.; Pinus spp.; Plantago spp.; Plectranthus barbatus; Polygala spp.; Polygonum spp.; Populus nigra; Porphyra umbilicalis, Portulaca oleracea; Potentilla spp.; Primula spp.; Prunus spp.; Punica granatum, Pygeum africanum, Pyrus malus, Quassia amara, Quercus spp.; Quillaja saponaria; Ranunculus ficaria; Raphanus spp.; Rhaponticum spp.; Ravensana aromatica, Rheum spp.; Rhodiola rosea; Ribes nigrum, Rosa spp.; Rosmarinus officinalis; Rubia tinctorium, Rubus spp.; Rumex occidentalis; Ruscus aculeatus; Saccharum officinarum, Satureia montana, Salix alba; Salvia spp.; Sambucus nigra; Schinus molle, Senna spp.; Serenoa repens; Silybum marianum, Solanum spp.; Solidago spp.; Sophora japonica; Sterculia spp.; Symphytium officinale; Syzygium aromaticum, Tagetes spp.; Tamarindus indica; Tanacetum spp.; Thea sinensis; Theobroma spp.; Thymus spp.; Tilia spp.; Trigonella foenum graecum, Triticum vulgare; Tropaeolum spp.; Tussilago farfara; Undaria spp.; Urtica dioica; Usnea spp.; Valeriana officinalis; Verbascum spp.; Verbena officinalis; Veronica spp.; Viola odorata; Viburnum spp.; Vinca minor; Vitis vinifera; Zea mays; and Zingiber officinale.

As mentioned earlier, basically every plant having one or more desired substances for one or more of a medicinal or cosmetic or dermatologic application can be used as raw material for the product according to the invention.

Whether used in a medicinal, cosmetic, or dermatologic application, in one embodiment, the reconstituted cannabis material can also contain one or more cannabinoids.

Cannabinoids that can be incorporated into the reconstituted plant material of the present disclosure include cannabidiol (CBD) and tetrahydrocannabinol (THC). THC contained in cannabis acts on specific receptors in the brain which lead to a feeling of euphoria and a relaxed state. CBD, on the other hand, also interacts with pain receptors in the brain but does not create the same euphoric feeling caused by THC. In accordance with the present disclosure, in one embodiment, THC can be applied to the reconstituted plant material of the present disclosure, CBD can be applied to the reconstituted plant material or, alternatively, both THC and CBD can be applied to the reconstituted plant material.

In addition to THC and CBD, various other cannabinoids can also be incorporated into the reconstituted plant material in accordance with the present disclosure. For instance, other cannabinoids contained in cannabis include cannabichromene, cannabinol, cannabigerol, tetrahydrocannabivarin, cannabidivarin, cannabidiolic acid, other cannabidiol derivatives, and other tetrahydrocannabinol derivatives. The above cannabinoids can be used singularly or in any combination and applied to the reconstituted plant material.

The cannabinoids described above can be applied to the reconstituted plant material using various different methods. For instance, in one embodiment, the cannabinoid, such as CBD, can be formulated into a powder that can be applied to the reconstituted plant material as an aqueous suspension. Alternatively, a cannabis oil extract may be obtained from raw cannabis plants. The oil extract may contain THC alone, CBD alone, or a combination of THC and CBD.

The following examples further describe and demonstrate embodiments that are within the scope of the present invention. The examples are given solely for the purpose of illustration, and are not to be construed as limitations of the present invention since many variations thereof are possible without departing from its spirit and scope.

### Examples

The following test method was used in obtaining the results in the examples below.

### Water Solubles Content

The sample of filler is ground into powder (using a IKA or RETSCHE-MUHLE grinder; mesh size: 1 mm).

A glass fiber filter (DURIEUX filter Nr 28, diam.=55 mm) is placed in a stainless steel pan. The tare of the pan+filter is then weighed (T, precision+/−1 mg). A 5000 mg (+/−200 mg) sample of ground filler is placed in the pan and precisely weighed (W1, precision+/−1 mg).

The ground filler is gently sprayed with water and the cup is installed into a lab percolator (RENEKA LC). Extraction is performed three times according to the pre-defined percolation settings. After percolation, the sample is cautiously washed with water and the pan is dried in an electric oven for 16 hrs at 100° C.

After washing, the pan is cooled in a dessicator during 15 min and its weight is measured (W3, precision+/−1 mg).

Dry weight of the ground sample used for Water solubles test (W2) is calculated as: W2−W1×(100−H)/100.

Finally, the ratio of Water solubles (%) in the dry finished product is calculated as follows:

$$WS ( \% ) = 1.15 \times ( ( \frac{W 2 - ( W 3 - T )}{W 2} ) \times 100 ) - 2.$$

Various different facial masks were made in accordance with the present disclosure using cannabis materials. The face masks are well suited for use in medicinal, cosmetic or dermatological applications. The masks were made according to the following method.

A mixture of cannabis leaves, cannabis flowers, cannabis seeds, cannabis seed shells or hulls, and cannabis hurds were mixed together and ground using a knife mill so as to obtain particles about 2 mm in size. The ground hemp material was then mixed with water at 70° C. for 45 minutes, in a hemp/water ratio of 1/10. The mixture was then pressed so as to separate the aqueous part (hemp fluid) from the insoluble part (hemp fibres, etc.). The fibrous fraction was combined with different pulp fibers and refined using disc refiners. More particularly, the raw hemp material was combined with hemp pulp to produce some samples and combined with softwood pulp to produce other samples. After being combined with the additional pulp fibers, the mixture was refined to 72° SR. The refined fibers were then fed through a papermaking process to produce a sheet-like product. The sheet-like product was then dried.

One sample was then treated with the water soluble extract obtained from the above process. The water soluble hemp extract was concentrated in an evaporator to a solid concentration of 50% and then coated on the reconstituted sheet using a size press.

During the papermaking process, a wet strength additive was added to the reconstituted sheets. Some of the sheets were treated with a humectant, namely glycerol. Each of the reconstituted sheets had a basis weight of 60 gsm.

The different samples were cut so as to form face-conforming masks. The following five samples were produced:

| Sample | A | B | C | D | E |
|---|---|---|---|---|---|
| Raw Hemp material | 60% | | 80% | | |
| Hemp pulp | 40% | | | | |
| Wood pulp | | | 20% | | |
| Glycerol | | 5% | | 5% | 5% |

| | |
|---|---|
| Extract | 13% |

| | |
|---|---|
| Wet strength additive | 0, .8% |
| (% in finish product) | |
| Base sheet basis | 60 g/m² |
| weight (g/m²) | |

All of the samples produced displayed excellent properties regarding mechanical strength, tactile feel, absorbency, softness, etc. The samples coated with glycerin displayed improved softness and flexibility.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A medicinal, cosmetic or dermatologic product comprising:
a sheet-like layer comprising a reconstituted cannabis material, the sheet-like layer having a shape configured to cover a portion of a user's body, the reconstituted cannabis material comprising extracted cannabis fibers comprising cannabis leaves, cannabis stems, cannabis buds, cannabis flowers, or mixtures thereof blended with web building fibers.

2. A medicinal, cosmetic or dermatologic product as defined in claim 1, wherein the reconstituted cannabis material comprises extracted cannabis fibers additionally comprising cannabis hurds.

3. A medicinal, cosmetic or dermatologic product as defined in claim 1, wherein the reconstituted cannabis material has been treated with a humectant.

4. A medicinal, cosmetic or dermatologic product as defined in claim 3, wherein the humectant comprises glycerol, propylene glycol or mixtures thereof and is present in the reconstituted cannabis material in an amount of about 2% to about 25% by weight.

5. (canceled)

6. A medicinal, cosmetic or dermatologic product as defined in claim 1, wherein the reconstituted cannabis material is formed from Cannabis sativa plant material containing less than 0.3% by weight tetrahydrocannabinol.

7. A medicinal, cosmetic or dermatologic product as defined in claim 1, wherein a plant extract has been applied to the reconstituted cannabis material, the plant extract forming a layer on the reconstituted cannabis material.

8. A medicinal, cosmetic or dermatologic product as defined in claim 1, wherein the reconstituted cannabis material contains water soluble cannabis components in an amount less than about 10% by weight.

9. A medicinal, cosmetic or dermatologic product as defined in claim 1, wherein the reconstituted cannabis material contains water soluble cannabis components in an amount from about 10% by weight to about 55% by weight.

10. (canceled)

11. A medicinal, cosmetic or dermatologic product as defined in claim 1, wherein the plant extract is impregnated in the layer of fibrous plant product.

12. A medicinal, cosmetic or dermatologic product as defined in claim 7, wherein the plant extract is applied to the reconstituted cannabis material as a fluid, gel, slurry or powder.

13. A medicinal, cosmetic or dermatologic product as defined in claim 10, wherein the plant extract comprises a blend of different plants.

14. A medicinal, cosmetic or dermatologic product as defined in claim 1, wherein the product comprises a wound dressing.

15. A medicinal, cosmetic or dermatologic product as defined in claim 1, wherein the product comprises a mask.

16. A medicinal, cosmetic or dermatologic product as defined in claim 15, wherein the mask includes eye openings and a mouth opening.

17. A medicinal, cosmetic or dermatologic product as defined in claim 1, wherein the reconstituted cannabis material includes from about 20% to about 50% by weight extracted cannabis leaves, from about 20% to about 50% by weight extracted cannabis buds and/or flowers, and from about 15% by weight to about 55% by weight web building fibers.

18. A medicinal, cosmetic or dermatologic product as defined in claim 1, wherein the web building fibers comprise delignified cellulose fibers, flax fibers, hemp fibers, abaca fibers, softwood fibers, hardwood fibers, bamboo fibers, coconut fibers, ramie fibers, jute fibers, or mixtures thereof.

19. (canceled)

20. A medicinal, cosmetic or dermatologic product as defined in claim 1, wherein the web building fibers comprise wood pulp fibers, the web building fibers being present in the reconstituted cannabis material in an amount greater than about 15% by weight and in an amount less than about 28% by weight.

21. A medicinal, cosmetic or dermatologic product as defined in claim 20, wherein the wood pulp fibers comprise a combination of softwood fibers and hardwood fibers.

22. A medicinal, cosmetic or dermatologic product as defined in claim 1, wherein the web building fibers comprise hemp pulp fibers.

23. A medicinal, cosmetic or dermatologic product as defined in claim 1, wherein the extracted cannabis fibers and the web building fibers are refined in an amount greater than about 40° SR and less than about 90° SR.

24. A medicinal, cosmetic or dermatologic product as defined in claim 1, wherein the reconstituted cannabis material has a basis weight of from about 25 gsm to about 100 gsm.

25. A method for producing a medicinal, cosmetic or dermatologic product comprising:
a) extracting one or more substances from at least one plant to obtain a plant extract, the at least one plant comprising cannabis leaves, cannabis stems, cannabis buds, cannabis flowers, or mixtures thereof;
b) separating the plant extract from a fibrous residue;
c) optionally refining the fibrous residue;
d) preparing a sheet-like product from the residue;
e) optionally concentrating and/or purifying and/or aromatizing the plant extract;
f) applying the plant extract to the sheet-like product.

26. (canceled)

27. (canceled)
